(19) United States
(12) Patent Application Publication
(10) Pub. No.: US 2010/0285152 A1
(43) Pub. Date: Nov. 11, 2010

(54) COMPOSITION FOR IMPROVING LIVER METABOLISM AND DIAGNOSTIC METHOD

(75) Inventors: Taru Pilvi, Helsinki (FI); Eero Mervaala, Espoo (FI); Riitta Korpela, Helsinki (FI)

Correspondence Address: SUGHRUE MION, PLLC, 2100 PENNSYLVANIA AVENUE, N.W., SUITE 800, WASHINGTON, DC, 20037, US

(73) Assignee: VALIO LTD., Helsinki (FI)

(21) Appl. No.: 12/811,266

(22) PCT Filed: Dec. 31, 2008
(86) PCT No.: PCT/FI2008/050796

§ 371 Date: Jun. 30, 2010

(30) Foreign Application Priority Data
Jan. 4, 2008 (FI) 20085007

(51) Int. Cl. A61K 38/16; A61K 33/06; A61P 3/00; A61P 3/04; A61P 3/10; C12Q 1/02; G01N 33/92

(52) U.S. Cl. 424/682; 514/6.7; 514/4.8; 514/6.9; 514/1.1; 435/29; 436/71

(57) ABSTRACT

There is a need to develop a treatment for metabolic syndrome, which is directed to maintaining healthy liver metabolism and not indirectly through weight loss. The present invention provides a composition comprising whey protein for supporting and improving liver metabolism, especially in connection with fatty liver. The composition can further comprise Ca and health improving components such as probiotics and prebiotics. The composition can be in the form of food, health food, food supplement or drugs. Furthermore, due to the complexity of choice of a valid biomarker and sample matrix, there is a special need to find out specific biomarkers for fatty liver and metabolic syndrome. This invention also relates to a diagnostic method for determining fatty liver on the basis of metabolomic profiling. Statistical modelling methods are used on the metabolomic profiles to identify the biomarkers.

### FIELD OF THE INVENTION

The present invention relates to a composition comprising whey protein for supporting and improving liver metabolism, especially in connection with fatty liver. Further, the present invention relates to a diagnostic method for determining fatty liver based on metabolomic profiling.

### BACKGROUND OF THE INVENTION

Abdominal obesity is closely related to the development of metabolic syndrome. Weight loss is today one of the few efficient treatments known to decrease metabolic syndrome. However, metabolic syndrome is not always a result of obesity. On the other hand not all obese people develop metabolic syndrome. The importance of fatty liver in the development of metabolic syndrome has only been understood in recent years.

It has been suggested that fat accumulation in the liver is the key feature distinguishing those individuals who develop metabolic syndrome from those who do not (Kotronen, A. and Yki-Järvinen, H., Fatty Liver. A Novel Component of the metabolic Syndrome, Arterioscler. Thromb. Vasc. Biol. 2007; (online Aug. 9, 2007). Once fatty, the liver is insulin resistant and over-produces major cardiovascular risk factors, such as C-reactive protein, (CRP), very low density lipoprotein (VLDL) and plasminogen activator inhibitor-1 (PAI-1) (Yki-Järvinen, H. Fat in the liver and insulin resistance, Ann Med. 2005; 37(5):347-356). At the moment improving insulin resistance through weight loss remains the cornerstone of therapy for non-alcoholic fatty liver disease (NAFLD). Furthermore, the means of preventing and treating hepatic fat accumulation are limited. Also, relatively little is known about the mechanisms that regulate the fatty liver. Consequently, there is a continuous need for better understanding the complexity of fatty liver on molecular level and finding out efficient and relevant means and specific compounds and/or agents to control, mitigate, alleviate or prevent the formation and/or release of fatty liver.

Whey protein in combination with calcium has been shown to attenuate body-weight and adipose-tissue gain in a model of diet-induced obesity (Pilvi, T. K., Korpela, R., Huttunen, M., Vapaatalo, H., Mervaala, E. M., High-calcium diet with whey protein attenuates body-weight gain in high-fat-fed C57B1/6J mice, Br. J. Nutrition, 007, 98: 900-907). Several studies have been made regarding e.g. whey protein in combination with calcium for weight management showing potential to reduce body fat or maintain lower weights by ‘switching off’ appetite after eating. The background art has proposed no indication of whey protein in combination with dietary calcium neither to accelerate weight loss under energy restriction nor consequently to have beneficial effects on liver fat reduction.

Several lipid metabolism-improving agents are known in the art such as glyceroglycolipid (Nisshin Sugar Manufacturing Co Ltd, JP 2005314256), an enzymatic digestion product of soybean protein (Fuji Oil Co Ltd, WO2003026685) and milk-derived basic protein or a basic peptide fraction (Snow Brand Milk Prod., JP 2002212097). Such agents and foods and drinks containing said agents are proposed to be useful for prevention and amelioration of lifestyle-related diseases such as fatty liver, hyperlipidemia, hypercholesterolemia, arteriosclerosis, obesity etc, without clear evidence of benefits.

Also an agent comprising three branched amino acids isoleucine, leucine and valine, as active ingredients for improving the expression of a gene involved in lipid metabolism is described in publication WO 2007/069744 (Ajinomoto Co., Inc.). Furthermore, WO 2006/070873 (Ajinomoto, Co., Inc.) describes food or beverage products exhibiting hypoadiponectinemia, hyperlipidemia, hypertension, angiopathy, fatty liver, hepatic fibrosis, or obesity preventive or therapeutic effect, comprising an adiponectin inducer or adiponectin secretion promoter comprising amino acids selected from leucine, isoleucine, valine, methionine, cysteine, alanine and mixtures thereof.

JP 2004300114 (Fuji Oil Co., Japan) described an oligopeptide mixture, obtained by decomposing soy-bean in the presence of endoprotease or exoprotease and processing by hydrophobic resin, which strongly controls the apolipoprotein B secretion from hepatocyte. According to the publication, the mixture is proposed for use in treating and preventing e.g. obesity, fatty liver, atherosclerosis, hypercholesterolemia, hypertriglyceridemia, diabetes, hypertension, chronic nephritis, liver cirrhosis, and obstructive jaundice.

Lipids are a highly diverse class of molecules with important roles as signaling and structural molecules in addition to serving as energy storage. It is crucial to identify the variety of lipid species accumulating in the liver in order to understand the complex process of hepatic insulin resistance. Puri et al. showed that in NAFLD in humans the level of triacylglycerides (TAG) and diacylglycerides (DAG) increased while total amount of phosphatidylcholines (PC) decreased (Puri P, Baillie R A, Wiest M M, Mirshahi F, Choudhury J, Cheung O, Sargeant C, Contos M J, Sanyal A J., A lipidomic analysis of nonalcoholic fatty liver disease, Hepatology, 2007, 46 (4) 1081-1090). Specifically, the accumulation of ceramides together with TAG and DAG seem to indicate the development of fatty liver.

Up-regulation of TAG and DAG, diacylphosphoglycerols and specific ceramide (CER) species and down-regulation of sphingomyelins (SM) has been seen in ob/ob mice (Yetukuri, L. Katajamaa, M., Medina-Gomez, G., Seppänen-Laakso, T., Vidal-Puig, A., Oresic, M., Bioinformatics strategies for lipidomics analysis: characterization of obesity related hepatic steatosis, BMC Systems Biol., 2007, 1:12). Furthermore, these earlier studies with genetically obese insulin resistant ob/ob mouse model do not show any mechanism of action and it is not a very human-like experimental model of lipidomic research.

There is a need to develop treatment for and prevention of the metabolic syndrome. This treatment and prevention should be directed at maintaining healthy liver metabolism and not indirectly through weight loss. Therefore, it is necessary to develop a direct treatment of fatty liver, which is directed at improve liver metabolism and to prevent the development of the metabolic syndrome.

Furthermore, due to the complexity of the choice of valid biomarker and sample matrix, there is a special need to find out specific biomarkers for fatty liver and metabolic syndrome. There is also a need to develop biomarkers that do not require unnecessary invasive sampling such as liver biopsy.

### BRIEF DESCRIPTION OF THE INVENTION

It is an object of the present invention to provide a composition comprising whey protein for prevention and/or treatment of fatty liver. Furthermore, another main object of the present invention is to better understand at molecular level the mechanism and key metabolites and their changes involved in fatty liver and to provide a specific treatment and biomarker for fatty liver.

The present invention provides a composition comprising whey protein for prevention and/or treatment of fatty liver.

Further, the present invention provides a method for supporting and improving liver metabolism, wherein the method comprises administering to the subject in need of such treatment a composition comprising whey protein. Still further, the present invention provides a method of diagnosing fatty liver in a subject, said method comprises determining the amount of at least one metabolite involved in the liver metabolism in a body sample taken from said subject, whereby an abnormal amount of said metabolite(s) indicates the status of liver metabolism.

The current invention provides a treatment for fatty liver, which is directed at improving the liver lipid metabolism and not indirectly through weight reduction.

There are also provided biomarkers for fatty liver and healthy liver lipid metabolism. The biomarkers can be measured from blood, serum or plasma and there is no need for biopsy of the liver.

The disclosed biomarkers provide a complete picture of the liver metabolism. Liver metabolism is a complex mechanism and the biomarkers should none the less provide a detailed picture of the condition of the liver metabolism. The diagnostic method disclosed herein provides exactly this.

### BRIEF DESCRIPTION OF THE DRAWINGS

FIG. 1 shows final body weight and body fat of obese control, weight reduced (casein=CPI and whey+Ca=WPI) and lean control mice (n=10/group). Bars represent mean±SEM values. Means without a common letter differ, p<0.05.

FIG. 2 shows PLS/DA score plot for metabolomic profiles in mouse liver for the different groups (Lean control; Obese; Weight loss (Casein=CPI); Weight loss (whey+Ca=WPI)).

FIG. 3 shows PLS/DA loadings. A Top 10 ranking lipids based on LV1 loadings, and top low ranking lipids. B Top 10 ranking lipids based on LV2 loadings, and top low ranking lipids.

FIG. 4 shows fold changes for 10 metabolites with highest and 10 metabolites with lowest level ratios between the lean and obese groups.

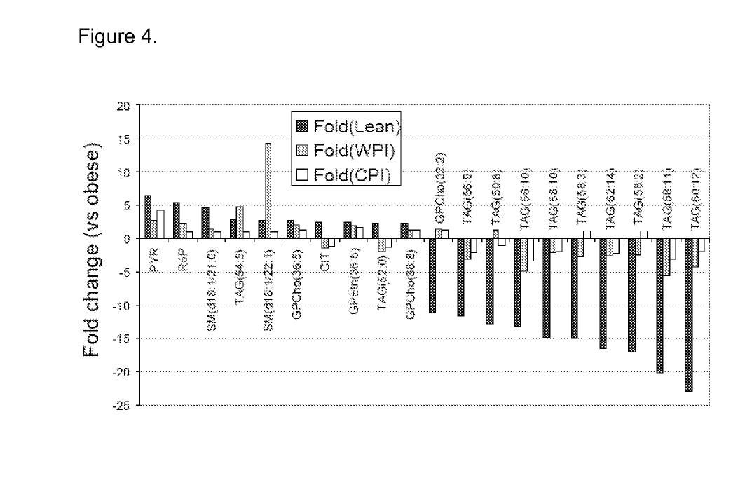

FIG. 5 shows Top 15 up-regulated and down-regulated metabolites in comparison with WPI and CPI groups.

FIG. 6 shows PLS/DA score plot for metabolomic profiles in serum (Lean control; Obese; Weight loss (Casein=CPI); Weight loss (whey+Ca=WPI)).

### DETAILED DESCRIPTION OF THE INVENTION

The present invention provides a composition comprising whey protein for improving and maintaining a healthy liver metabolism and is useful in treatment for and/or prevention of fatty liver. Fatty liver is closely related to obesity and metabolic syndrome and thus also to insulin resistance and diabetes type II. Treatment of fatty liver is therefore useful in patients suffering from metabolic syndrome. Metabolic syndrome has traditionally been treated with programs or medicaments aiming at weight loss of the patient. The composition according to the present invention enables a treatment that is not directed at weight loss but towards the improvement of the liver metabolism. Therefore, the composition is useful for treating normal weight or slim patients suffering from fatty liver and metabolic syndrome.

The composition according to the present invention can preferably also contain calcium. The combination of whey protein and calcium has been found especially useful in the treatment of fatty liver. The composition may further contain other health improving and sustaining components such as probiotics and prebiotics.

The current invention is based on the surprising findings that a whey protein diet significantly improves the liver metabolite profiling. The improvement of the metabolite profile shows that the whey protein diet acts directly on the well-being of the liver. An improved metabolite profile is very important in the treatment and prevention of fatty liver. The current invention therefore provides a method for restoring and maintaining healthy metabolism of the liver.

A healthy liver metabolism is essential in prevention and treatment of metabolic syndrome. The current invention provides a treatment for all patients suffering from metabolic syndrome, since the treatment is directed to the liver lipid metabolism directly and not to weight loss. Therefore, a subject group suffering from metabolic syndrome but not suffering from obesity can now be treated with the method provided herein.

The composition according to the current invention improves the liver metabolite profile. It is therefore important that the lipid metabolism can be monitored prior to and especially during such a treatment. The current invention thus also provides a method for monitoring liver lipid metabolism.

Another aspect of the current invention is to monitor liver metabolism with metabolomic biomarkers from a body sample. It is thus provided a method of diagnosing fatty liver in a subject, wherein said method comprises determining the amount of at least one metabolite involved in the liver metabolism in a body sample taken from said subject, whereby an abnormal amount of said metabolite(s) indicates the status of the liver metabolism. Preferably the body sample is a blood sample and the amounts of several metabolites are determined simultaneously. It was surprisingly found that liver metabolism (fatty liver) could be monitored and followed from serum isolated from blood samples without the need of biopsy sampling. Thus, there is provided a method for determining the status of the fatty liver by measuring metabolomic biomarkers from a blood sample. The method provided herein can be used for determining fatty liver or for monitoring the development of the disease during the treatment.

The metabolomic biomarkers can be established by collecting a lipidomic profile, a water soluble metabolite profile or a combination of a lipidomic and a water soluble metabolite profile.

Metabolomic profiling is a large-scale study of non-water-soluble (lipids) and water soluble metabolites. The metabolomic profiles can be obtained by technologies such as electrospray ionization (ESI(+/−)), mass spectrometry (MS), liquid chromatography coupled to mass spectrometry (LC/MS) and comprehensive two-dimensional gas chromatography coupled to a high speed time-of-flight mass spectrometry (GC×GC-TOF). Relationships between the metabolites are characterized typically by multivariate methods. This enables analysis of several or even numerous metabolites simultaneously from a single sample to obtain a “lipid profile”, “water soluble metabolite profile” or a “metabolomic profile” (i.e. a combination of lipid and water soluble metabolites). These results may then be used to identify a metabolic profile typical to fatty liver using statistical modeling methods.

Primary metabolites are a selected set of metabolites, which are the key metabolites in the energy metabolism pathways, like TCA-cycle and pentose phosphate pathway.

Combining the lipidomic and water soluble metabolite profiles provides an accurate and reliable biomarker for fatty liver.

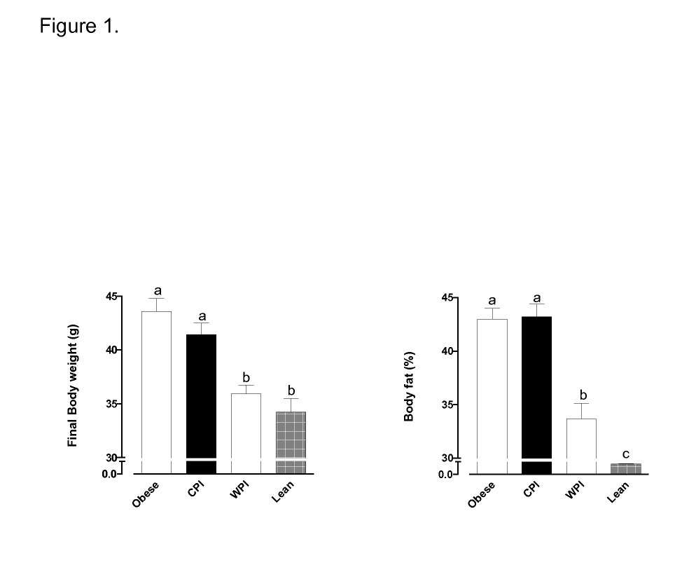

It has now been surprisingly found that by modulating the protein source and calcium content of the weight loss diet the comprehensive lipidomic and primary metabolite profile can be significantly improved (Example 2). Furthermore, whey protein and calcium significantly accelerated weight and fat loss and decreased fat absorption during energy restriction (Example 1, FIG. 1).

Furthermore, it has been surprisingly shown that weight loss improved the liver lipid profile with a clear indication of significant changes in triacylglycerol, phospholipids and ceramide content of the liver. When the weight loss was accompanied with a whey protein diet the improvement in the liver metabolite profile was even more pronounced than weight loss without whey protein.

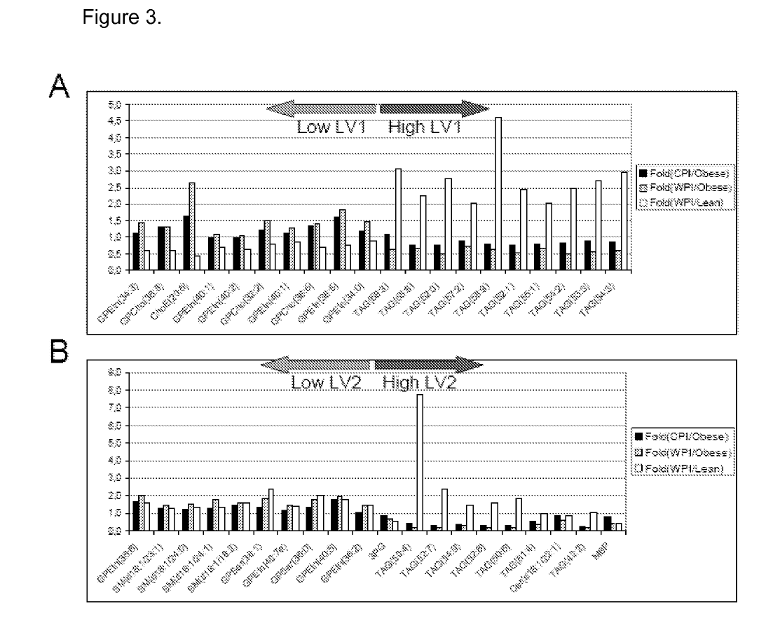

The major changes in the liver lipid profile associated with high-fat diet induced obesity were the increased amount of TAG and decreased amount of major phospholipids, such as phosphatidyletanolamines and phosphatidylcholines (Example 2, FIG. 3 A). Some of the TAG species were increased even 10 to 20-fold in the Obese group in comparison with the Lean group. Also, certain ceramides were among the species with the highest increase calculated by fold change.

The present findings show that weight loss with or without a whey protein diet was associated with decreased level of TAG and increased level of sphingomyelins, cholesterol esters and phosphatidylserines. The metabolite changes that best separated the weight loss groups from the Obese were the reduction in specific TAG and ceramide species and increase in sphingomyelins, cholesterol esters and phosphatidylserines (FIG. 3 B).

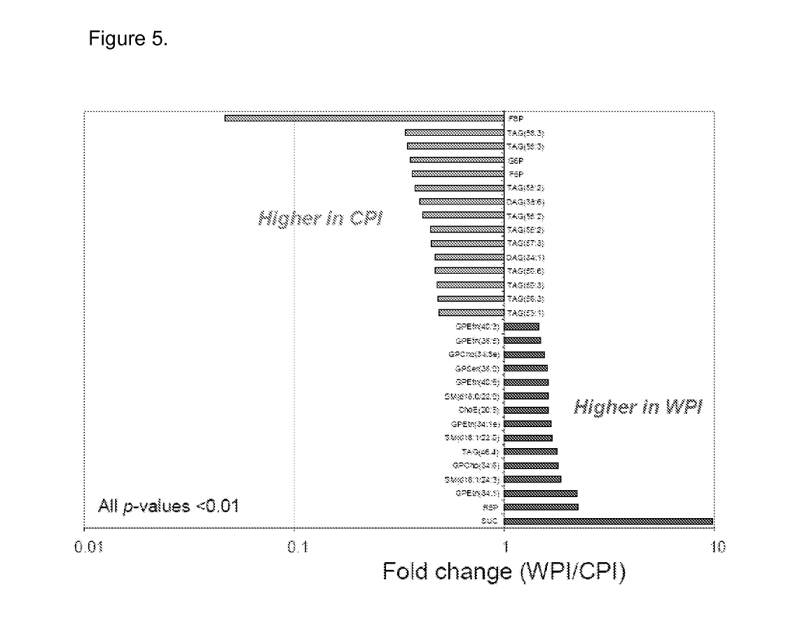

However, when the weight loss was accompanied with a whey protein diet (Whey+Ca=WPI) the level of several phospholipids species like phosphatidyletanolamines and sphingomyelins increased and TAG and glycolytic metabolites decreased in comparison with weight loss on Control diet (CPI) (FIG. 5).

Whey+Ca (WPI) treatment significantly modulated primary metabolism. Direct comparison of metabolite levels between the weight loss groups revealed surprisingly, that weight loss with WPI diet was associated with increased levels of TCA cycle and pentose phosphate pathway metabolites. WPI diet also increased the level of several phospholipids species like phosphatidyletanolamines and sphingomyelins and decreased TAG and glycolytic metabolites in comparison with weight loss on Control diet (CPI) (FIG. 5).

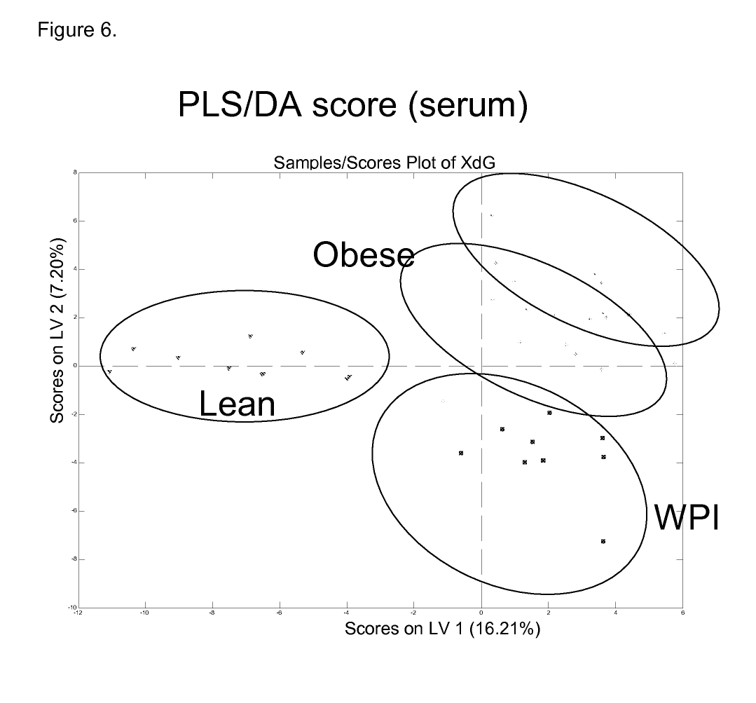

Surprisingly, Whey+Ca (WPI) diet had remarkable effect also on the serum metabolite profile (Example 3, FIG. 6). Thus, there is a strong indication of a valid non-invasive method for comprehensive metabolic profiling and modelling the specific physiological state. A method that does not require that a biopsy sample of the liver be taken is here referred to as a “non-invasive” method. For example a blood sample is here considered a non-invasive sampling method. A blood sample can be collected during a routine health inspection and can be performed in any medical laboratory. In the present invention “whey protein” refers to whey-derived protein, whey-derived peptide fraction, whey-derived protein isolate, whey-derived protein hydrolysate, whey components and/or combinations or mixtures thereof. Whey protein is a collection of globular proteins that can be isolated from whey, a by-product of cheese manufactured from cow's milk. It is typically a mixture of β-lactoglobulin (˜65%), α-lactalbumin (˜25%), and serum albumin (˜8%). Whey protein contains high levels of both essential and non-essential amino acids.

On a commercial scale, several methods are available for production of whey protein-rich products, for the removal of whey proteins from whey and for the purification of the major and minor whey proteins. Whey-derived protein isolate typically comprises bovine serum albumin, α-lactoglobulin, β-lactoglobulin, κ-casein fragment(s), lactoferrin etc.

According to the present invention, the composition comprising whey protein can be in the form of food, health food and drugs. Furthermore, compositions and applications can be produced in a form that allows them to be consumed as a convenient part or a supplement, for example, of the everyday diet.

Accordingly, the composition of the invention can be administered orally as such, i.e., in the form of a tablet, capsule or powder. In addition, the composition of the invention can be administered orally as a food or nutritional product, such as dairy product, or as a pharmaceutical product.

The term “food product” is intended to cover all consumable products that can be solid, jellied or liquid, and to cover both ready-made products and products which are produced by using the composition of the invention alone or in combination with conventional food products or ingredients. Food products can for instance be products of dairy industry or beverage industry.

Accordingly, the composition according to the present invention can be added to a food product or medicament during the manufacture of the food or pharmaceutical product. The composition according to the present invention can also be added to the finished food product. The food products in question thus have the desired effect on fatty liver and thus also on metabolic syndrome.

The form of each of the food product, food material, and/or the pharmaceutical products, and the animal feed is not particularly limited. Examples of suitable food and/or nutritional products include dairy products, drinks, juices, soups or children's foods.

The composition and the products of the invention are primarily suitable for use for human adults and infants. The positive effects of the products are also beneficial to animals, especially pets and production animals. Examples of these include dogs, cats, rabbits, horses, cows, pigs, goats, sheep and poultry.

The following examples illustrate the present invention. The examples are not to be construed to limit the claims in any manner whatsoever.

### Example 1

### Body Weight and Fat Content

Eight-week old male C57Bl/6J mice (n=40, Harlan, Horst, The

Netherlands) were housed five in a cage in a standard experimental animal laboratory, illuminated from 6.30 a.m. to 6.30 p.m., temperature 22±1° C. The mice had free access to feed and tap water. After a one-week acclimatisation period on a normal chow diet (Harlan Tekland 2018, Harlan Holding, Inc, Wilmington, Del., USA) thirty mice (25.5±0.3 g) were put on a high-fat diet (60% of energy from fat; protein 23.4%, carbohydrate 26.6%, fat 35.3%, fiber 6.5%; protein=Alacid 714 acid casein, NZMP, Auckland, New Zealand). Ten remaining mice continued on normal chow diet (ad libitum) throughout the study (Lean control group). After the weight gain period of 14 weeks on high-fat diet one group of mice (Obese group, n=10) was sacrificed and the remaining mice were put on an energy restriction diet for 7 weeks. During the energy restriction period the mice were given 70% of the energy they ate during the ad libitum feeding. At the beginning of the energy restriction period the body weight matched mice were divided into two groups (WPI and CPI). WPI group received high-fat diet (protein 23.1%, carbohydrate 26.2%, fat 35.0%, fiber 6.5%) with 1.8% CaCO₃ and all protein (18% of energy) from whey protein isolate (Alacen™ 895, NZMP, Auckland, New Zealand). The CPI group continued with the same high-fat diet (60% of energy from fat; Alacid 714 protein 23.4%, carbohydrate 26.6%, fat 35.3%, fiber 6.5%) as during the weight gain period.

The body weight was monitored weekly during the weight gain period and twice per week during the energy restriction period. The consumption of feed was monitored daily. The body fat content was analysed by dual-energy x-ray absorptiometry (DEXA, Lunar PIXImus, GE Healthcare, Chalfont St. Giles, UK) at the end of the weight gain and energy restriction periods.

Results: Whey protein and calcium accelerated weight and fat loss and decreased fat absorption during energy restriction. At the end of the weight gain period the high-fat fed mice weighed significantly more than the chow fed control mice (41.5±1.0 g vs. 34.3±1.3 g, p<0.001) (FIG. 1). The obese mice also had significantly more fat tissue than the lean controls (43.1±1.0% vs. 25.5±1.0, p<0.001). The 7-week energy restriction reduced the body weight in the WPI group, to the level of lean controls (p<0.001 vs. Obese and p>0.05 vs Lean) but the decrease in body weight was not statistically significant in the CPI group. WPI also reduced the fat pad weights more than the weight loss on CPI diet.

### Example 2

### Metabolomic Profiling

Sample preparation: At the end of the treatment period the mice were rendered unconscious with CO₂/O₂ (95%/5%), and decapitated. The livers were removed, washed with saline, blotted dry and weighted. The tissue samples were snap-frozen in liquid nitrogen and stored at −80° C. until assayed.

Lipids from the lipidomic analysis were named according to Lipid Maps (http://www.lipidmaps.org). For example, lysophosphatidylcholine with 16:0 fatty acid chain was named as monoacyl-glycerophosphocholine GPCho(16:0/0:0). In case the fatty acid composition was not determined, the-total number of carbons and double bonds was marked. For example, a phosphatidylcholine species GPCho(16:0/20:4) is represented as GPCho(36:4). However, GPCho(36:4) could also represent other molecular species, for example GPCho(20:4/16:0) or GPCho(18:2/18:2).

Lipidomics: Liver tissue samples (n=10/group), 10 μl of an internal standard mixture containing GPCho(17:0/17:0), GPEtn(1p:0/17:0) (glycerophosphoethanolamines), GPCho(17:0/0:0), Cer(d18:1/17:0) (ceramides) and TG(17:0/17:0/17:0) (triacylglycerol) and 200 μl of chloroform:methanol (2:1) were homogenized in 2 ml Eppendorf tubes with a ball mill by using glass balls. Sodium chloride solution (0.15 M, 50 μl) was added and the samples were vortexed for 2 minutes. After 1 hour extraction time the samples were centrifuged for 3 min at 10000 rpm and 100 μl aliquots of the lower layers were taken to glass inserts and mixed with 10 μl of a mixture containing GPCho(16:1/16:1-D6), GPCho(16:1/0:0-D3) and TG(16:0/16:0/16:0-13C3).

Liver tissue extracts were examined by a Q-T of Premier mass spectrometer by introducing the sample through an Acquity UPLC™ system equipped with an Acquity UPLC™ BEH C18 1×50 mm column with 1.7 μm particles. The temperature of the column was 50° C. The solvent system consisted of water (1% 1M NH₄Ac, 0.1% HCOOH) and acetonitrile/isopropanol (5:2, 1% 1M NH₄Ac, 0.1% HCOOH) and the flow rate was 0.200 ml/min. The compounds were detected by using electrospray ionization in positive ion mode (ESI+). Data was collected at m/z 300-1200 with a scan duration of 0.2 s. The source and desolvation temperatures were 120° C. and 250° C., respectively.

Data was processed using MZmine software version 0.60 (Katajamaa and Oresic, 2005), and metabolites were identified using internal spectral library or with tandem mass spectrometry (Yetukuri et al., 2007).

Primary metabolites: Twenty mg of frozen liver tissue (n=10/group) was weighted into Eppendorf tubes and 200 μl of methanol (−80° C.) and 10 μl of ¹³C labeled internal standard was added. Sample was homogenized with Micro Dismembrator S (Sartorius, Germany) by using glass beads (0.5-0.75 mm) and 3000 rpm for three minutes. Homogenized samples were boiled immediately in 80° C. for three minutes and at 10000 rpm for 5 minutes. Supernatant was collected and evaporated to dryness under stream of nitrogen. Samples were reconstituted in 100 μl of ultrapure water.

The liver extracts were analyzed with HPLC-MS/MS method for quantitative analysis of phosphorous and TCA-cycle compounds. The system consisted of HT-Alliance HPLC (Waters, Milford, Mass.) working at high pH. The analytes were resolved by anion exchange chromatography combined with post column ASRS Ultra II 2 mm ion suppressor (Dionex, Sunnyvale, Calif.) and detected with Quattro Micro triple quadrupole mass spectrometry (Waters, Milford, Mass.) operating in electrospray negative ion mode. The analytical column was IonPac AS11 (2×250 mm, Dionex, Sunnyvale, Calif.) and guard column IonPac AG11 (2×50 mm, Dionex, Sunnyvale, Calif.). Flow rate was 250 μl/min and injection volume 5 μl. The temperature of the column was 35° C. and autosampler 10° C. The gradient mixture of water (99-52%) and 300 mM NaOH (1.0-48%) was used.

The compounds were detected in Multiple Reaction Monitoring (MRM) mode for optimal sensitivity and selectivity. A small aliquot of ¹³C-labelled metabolites from yeast fedbatch cultivation was used as an internal standard. Hexose phosphates (glucose-6-phosphate (G6P), fructose-6-phosphate (F6P), mannose-6-phosphate (M6P) and 6-glucose-1-phosphate (6G1P)), pentose phosphates (ribose-5-phosphate (R5P) and ribulose-5-phosphate (R15P)), fructose bisphosphate (FBP), glycerate-2-phosphate (G2P) and 3-phosphoglycerate (3PG), phosphoenolpyruvate (PEP), 6-phosphogluconate (6PG), succinate (SUC), malate (MAL), α-ketoglutarate (AKG), oxaloacetate (OXA), citrate (CIT), iso-citrate (ICI), glyoxylate (GLY) and pyruvate (PYR) were quantitatively measured.

Data was processed with MassLynx 4.1 software and internal calibration curves were calculated on the basis of the response of ¹²C analyte and ¹³C labelled analogue.

Data analysis: Partial least squares discriminant analysis (PLS/DA) and PLS analysis were utilized as a modeling methods for clustering and regression of lipidomics and primary metabolites data. PLS/DA is a pattern recognition technique that correlates variation in the dataset with class membership. The resulting projection model gives latent variables (LVs) that focus on maximum separation (“discrimination”). Contiguous blocks cross-validation method and Q² scores were used to develop the models. The VIP (variable importance in the projection) values were calculated to identify the most important molecular species for the clustering of specific groups. Multivariate analyses were performed using Matlab version 7.2 (Mathworks, Natick, Mass.) and the PLS Toolbox version 4.0 Matlab package (Eigenvector Research, Wenatchee, Wash.). Comparisons between levels of selected molecular species were performed using the two sided t-test.

Correlation of metabolites with blood glucose was performed by using PLS regression analysis with contiguous block cross-validation.

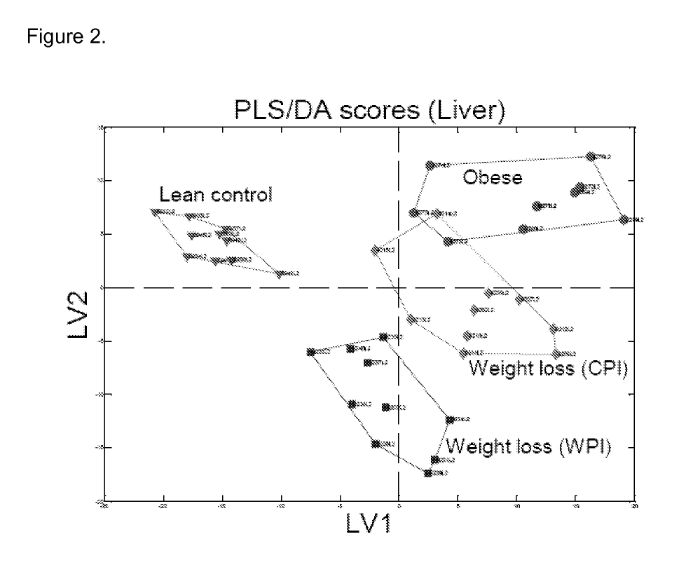

Results: The lipidomic and primary metabolite profile is significantly altered by diet-induced obesity and weight loss. Lipidomic profile included 391 identified lipid species and the primary metabolite analysis led to quantification of 13 metabolites (G6P, F6P, M6P, FBP, 3PG, R5P, SUC, MAL, CIT, PYR, PEP, 6PG, FUM). PLS-DA analysis of combined lipidomic and primary metabolites data revealed marked differences between the groups (FIG. 2). Specifically, the first latent variable (LV1) revealed changes related to the differences in body weight, while the differences along second latent variable (LV2) were more specific to the weight loss and diet effect. The effect of the Whey+Ca (WPI) diet was clearly stronger than the effect of weight loss as such and brought the group closer to the Lean controls. However, the treatment led to marked metabolic changes distinct from the Lean controls.

Obesity increased the amount of TAG and decreased the level of major phospholipids. The major changes associated with high-fat diet induced obesity were the increased amount of TAG and decreased amount of major phospholipids, such as phosphatidyletanolamines and phosphatidylcholines (FIG. 3 A). Some of the TAG species were increased even 10 to 20-fold in the Obese group in comparison with the Lean group. Also certain ceramides were among the species with the highest increase. Obesity induced fatty liver was not as much associated with decreased amount of metabolites. The biggest negative fold change was observed in pyruvate (PYR) and ribose-5-phosphate (R5P) followed by certain sphingomyelins and other phospholipids species.

Weight loss was associated with decreased level of TAG and increased level of sphingomyelins, cholesterol esters and phosphatidylserines. The metabolite changes that best separated the weight loss group from the Obese were the reduction in specific TAG and ceramide species and increase in sphingomyelins, cholesterol esters and phosphatidylserines (FIG. 3 B).

Whey+Ca (WPI) treatment significantly modulated primary metabolism. Direct comparison of metabolite levels between the weight loss groups revealed surprisingly, that weight loss with WPI diet was associated with increased levels of TCA cycle and pentose phosphate pathway metabolites. WPI diet also increased the level of several phospholipids species like phosphatidyletanolamines and sphingomyelins and decreased TAG and glycolytic metabolites in comparison with weight loss on Control diet (CPI) (FIG. 5).

### Example 3

### Metabolomic Profiling of Serum

Sample preparation: Serum samples were analysed by adding an aliquot (10 μl) of an internal standard mixture containing equal amounts of, internal standards (GPCho(17:0/0:0), GPCho(17:0/17:0), GPEtn(17:0/17:0), GPGro(17:0/17:0)[rac], Cer(d18:1/17:0), GPSer(17:0/17:0) and GPA(17:0/17:0) from Avanti Polar Lipids and MG(17:0/0:0/0:0)[rac], DG(17:0/17:0/0:0)[rac] and TG(17:0/17:0/17:0) from Larodan Fine Chemical) and 0.05 M sodium chloride (10 μl) were added to serum samples (10 μl) and the lipids were extracted with chloroform/methanol (2:1, 100 μl). After vortexing (2 min), standing (1 hour) and centrifugation (10000 RPM, 3 min), the lower layer was separated and a standard mixture containing 3 labeled standard lipids was added (10 μl) to the extracts. The standard solution contained 10 μg/ml (in chloroform:methanol 2:1) GPCho(16:0/0:0-D3), GPCho(16:1/16:1-D6) and TG(16:0/16:0/16:0-¹³C3), all from Larodan Fine Chemicals. The sample order for LC/MS analysis was determined by randomization.

Lipid extracts were analysed on a Waters Q-T of Premier mass spectrometer combined with an Acquity Ultra Performance LC™. The column, which was kept at 50° C., was an Acquity UPLC™ BEH C18 10×50 mm with 1.7 μm particles. The binary solvent system included A. water (1% 1 M NH₄Ac, 0.1% HCOOH) and B. LC/MS grade (Rathburn) acetonitrile/isopropanol (5:2, 1% 1 M NH₄Ac, 0.1% HCOOH). The gradient started from 65% N35% B, reached 100% B in 6 min and remained there for the next 7 min. The total run time including a 5 min re-equilibration step was 18 min. The flow rate was 0.200 ml/min and the injected amount 0.75 μl. The temperature of the sample organizer was set at 10° C.

The lipid profiling, data procession and identification of lipids was carried out in the similar manner as Example 2.

### Analysis of Water-Soluble Metabolites by GC×GC-TOF

A broad screening of water-soluble metabolites was conducted by a comprehensive two-dimensional gas chromatography coupled to a high speed time-of-flight mass spectrometry (GC×GC-TOF) (Welthagen W, Shellie R, Spranger J, Ristow M, Zimmermann R, Fiehn O, Comprehensive two-dimensional gas chromatography-time-of-flight mass spectrometry (GC×GC-TOF) for high resolution metabolomics: biomarker discovery on spleen tissue extracts of obese NZO compared to lean C57BL/6 mice. Metabolomics 2005; 1:65-73) The instrument used was a Leco Pegasus 4D GC×GC-TOF with Agilent 6890N GC from Agilent Technologies, USA and CombiPAL autosampler from CTC Analytics AG, Switzerland. Modulator, secondary oven and time-of flight mass spectrometer are from Leco Inc., USA. The GC was operated in split mode (1:20) using helium as carrier gas at 1.5 ml/min constant flow. The first GC column was a relatively non-polar RTX-5 column, 10 m×0.18 mm×0.20 μm, and the second was a polar BPX-50, 1.10 m×0.10 mm×0.10 μm. The temperature programme was as follows: initial 50° C., 1 min ->280° C., 7° C./min, 1 min. The secondary oven was set to +30° C. above the primary oven temperature. The second dimension separation time was set to 3 seconds. The mass range used was 40 to 600 amu and the data collection speed was 100 spectra/second. A commercial mass spectral library, Palisade Complete 600K, was used for identifying metabolites.

Results: 129 metabolites were identified (GC×GC-TOF platform), and 537 lipids identified from serum (HPLC/MS (ESI+). PLS-DA analysis of lipidomic and primary metabolites data revealed marked differences between the groups, with a remarkable effect of Whey+Ca (WPI) diet on serum metabolite profile as shown in FIG. 6.

1. A composition comprising whey protein for prevention and/or treatment of fatty liver.

2. The composition according to claim 1, wherein the prevention and/or treatment of fatty liver is connected to one or more of the following obesity, metabolic syndrome, type II diabetes and insulin resistance.

3. The composition according to claim 1, wherein the composition is further containing calcium.

4. The composition according to claim 1, wherein the composition further comprises probiotics and/or prebiotics.

5. The composition according to claim 1, wherein the composition is in the form of a functional food.

6. The composition according to claim 5, wherein the functional food is in the form of dairy products, drinks, juices, soups or children's food.

7. The composition according to claim 1, wherein the composition is in the form of a health promoting natural product.

8. The composition according to claim 7, wherein the health promoting natural product is in the form of pills, tablets, powders or mixtures.

9. A method for supporting and improving liver metabolism, wherein the method comprises administering to the subject in need of such treatment a composition comprising whey protein.

10. A method of diagnosing fatty liver in a subject, wherein said method comprises determining the amount of at least one metabolite involved in the liver metabolism in a blood sample taken from said subject, whereby an abnormal amount of said metabolite(s) indicates the status of liver metabolism.

11. The method of claim 10, wherein the amounts of several of said metabolites are determined simultaneously.

12. The method according to claim 10, characterized in that the metabolites have been established by collecting a lipid profile, a water soluble metabolite profile or a combination of a lipid and water soluble metabolite profile.

13. The method according to claim 12, characterized in that statistical modelling methods are used on the collected profile to identify abnormal amounts of said metabolite(s).

14. The method according to claim 12, characterized in that said profiles are collected using techniques such as gas or liquid chromatography coupled to mass spectrometry.

15. The method according to claim 10, characterized in that said metabolites are measured from a serum or plasma sample isolated from the blood sample.

16. The method according to claim 10, characterized in that the method is used for monitoring the development of fatty liver during treatment of said disease.

17. The method according to claim 16, characterized in that said treatment comprises administering a composition comprising whey protein for prevention and/or treatment of fatty liver to a patient in need thereof.
